(19) United States
(12) Patent Application Publication
(10) Pub. No.: US 2018/0193587 A1
(43) Pub. Date: Jul. 12, 2018

(54) Method of delivering subliminal messages

(71) Applicant: Arthur Glass, Staten Island, NY (US)

(72) Inventor: Arthur Glass, Staten Island, NY (US)

(21) Appl. No.: 15/400,969

(22) Filed: Jan. 7, 2017

(51) Int. Cl. A61M 21/00; G06F 3/16

(52) U.S. Cl. CPC A61M 21/00; A61M 2021/0027; G06F 3/16

(57) ABSTRACT

This invention relates to a method of delivering subliminal messages to a user for providing positive benefits by using the subliminal programming app. The frequencies of the subliminal messages are such that which cannot be heard by human ear. Further, the subliminal messages can be audio subliminal messages and the positive benefits can be relieving the user from various diseases, improving the user's cognition health, relaxing the user, relieving anxiety, relieving the stress, relieving pain, relieving grief, helping to quit alcohol, helping to quit smoking, improving sexual health, increasing memory, improving weight loss results, controlling the diabetes, controlling the weight gain, curing the diabetes, increasing intelligence, making leaning in any field happening faster, learning a new foreign language bringing behavioral changes, or similar type of positive benefits.

### (1) FIELD OF THE INVENTION

The invention particularly relates to a method to inject unrecognized low frequency audio (subliminal messages) within an installed device by using the subliminal programming app. These subliminal messages will reach the end user who is performing any kind of audio visual activity on the said device. This would help in relieving the brain and creating new neural pathways which will eventually improve the user's cognition health by relaxing and relieving anxiety, stress, pain etc. and would lead to many other health benefits.

The invention is a computer-implemented method for delivering audio messages to the end-user via an installable program.

### (2) BACKGROUND OF THE INVENTION

A subliminal message is a signal or message designed to pass below (sub) the normal limits of perception. For example it might be inaudible to the conscious mind (but audible to the unconscious or deeper mind) or might be an image transmitted briefly and unperceived consciously and yet perceived unconsciously. It is suspected and usually tested in psychological studies that subliminal messages gain their potential ability to influence from the fact that they may be able to circumvent the conscious awareness and its critical functions. This route to influence or persuasion would be akin to auto-suggestion or hypnosis wherein the subject is encouraged to be (or induced to be) relaxed so that suggestions are directed to deeper parts of the mind; some observers have argued that the unconscious mind is incapable of critical refusal of hypnotic or subliminal suggestions.

A number of types of devices and methods are for delivering subliminal messages are available in prior art. A prior art document, WO1994026063 discloses a method of displaying a subliminal message to an observer upon a rasterised display, said method comprising the steps of displaying a video source material comprised of a plurality of sequential fields, displaying a subliminal message for a portion of one of said fields, and immediately following display of said subliminal message, erasing said subliminal message from display prior to recommencement of uninhibited display of said video source material. Another prior art document, US20120046099 discloses methods of passing subliminal messages to a subconscious mind. In particular, the methods relate to rapidly covering and uncovering a message, to increasing the speed, and to improving time length of the execution. Yet another prior art document, U.S. Pat. No. 6,122,322 discloses a method and to a system for detecting a first context change between two frames. When a second context change between a further two frames occurs within a predetermined time interval, the frames accommodated within the two context changes are defined as a subliminal message. An alarm is sent to an observer upon detection of a subliminal message. Yet another prior art document, U.S. Pat. No. 5,784,124 discloses a method of behavior modification involves having a patient view supraliminal video messages superimposed upon an underlying video presentation. Yet another prior art document, U.S. Pat. No. 5,644,363 discloses an apparatus for superimposing subliminal and non-subliminal video messages on a video signal to generate a combined video signal. Another prior art document, U.S. Pat. No. 5,245,666 discloses a method and system to allow a message to be presented to the user through an earphone at a subliminal level. The system uses the sound, measured at the ear, as the cover sound. This means that an affirmative message may be chosen, recorded, and used, subliminally, within seconds. It is designed to be easy to use, portable and is non-intrusive to the everyday flow of life. Yet another prior art document, WO1990012470 discloses a method and apparatus for processing subliminal audio and video messages for television signals. Yet another prior art document, U.S. Pat. No. 5,221,962 discloses a method and apparatus for presenting subliminal visual and/or audio messages which allows user verification of message content and presence, as well as proper adjustment of message obviousness while accounting for ambient conditions and user sensitivities. Yet another prior art document, U.S. Pat. No. 5,159,703 discloses a technique for producing and transmitting a subliminal presentation which is inaudible to the listeners(s), yet is perceived and demodulated (decoded) by the ear for use by the subconscious mind. Yet another prior art document, U.S. Pat. No. 5,215,468 discloses a method and apparatus for, during physical activity, motivating a user to change the rate of his or her physical activity while receiving subliminally changing audio stimuli, the physical activity being synchronized to the audio stimuli. Yet another prior art document, U.S. Pat. No. 5,017,143 discloses a method and apparatus to produce more effective visual subliminal communications. Graphic and/or text images, presented for durations of less than a video frame, at organized rhythmic intervals, the rhythmic intervals intended to affect user receptivity, moods or behavior. Subliminal graphic images have translucent visual values locally dependent on background values in order to maintain desired levels of visual contrast. Yet another prior art document, U.S. Pat. No. 4,777,529 discloses an auditory subliminal programming system includes a subliminal message encoder that generates fixed frequency security tones and combines them with a subliminal message signal to produce an encoded subliminal message signal which is recorded on audio tape or the like.

However, above mentioned references and many other similar references have one or more of the following shortcomings or limitations: (i) User or listener has to play only specific audio/video files; (ii) subliminal messages delivered only with the videos; (iii) subliminal messages deliver in assisting to play games on computer; (iv) subliminal frame containing subliminal message inserting between the frames; (v) subliminal message delivering while the user is watching only the television; (vi) the user has to play repetitively same audio/video files and probably the user get bored; and (vii) only focus on specific type of benefits like only for the fitness improvement.

### (3) SUMMARY OF THE INVENTION

In the view of the foregoing disadvantages inherent in the known types of devices and method for delivering subliminal messages now present in the prior art, the present invention provides a novel method for delivering subliminal messages to the user. As such, the general purpose of the present invention, which will be described subsequently in greater detail, is to provide a new and improved method for delivering subliminal messages to the user for providing positive benefits which has all the advantages of the prior art and none of the disadvantages.

The object of the invention is to provide a method of delivering “audio” subliminal messages to a user. It would include following steps: (i) Downloading a subliminal programming app to an object device; (ii) Log-in to the subliminal programming app on the object device; (iii) The user has the option to select one of the categories from the list of positive benefits; (iv) Using the subliminal programming app as a portal to start an activity; wherein the activity is selecting from the starting web-surfing, start playing video games, for selecting choice of a website to play audio and/or video contents, for selecting choice of an mobile app to play audio and/or video contents, or combination thereof; and (v) Delivering the subliminal messages by the subliminal programming app on the background or on the secondary track to the user while he/she is performing the activity.

Another object of the invention is to provide the method of delivering subliminal messages to a user for providing positive benefits, wherein the subliminal programming app includes internet website based applications, server based applications, mobile applications or combination thereof.

Another object of the invention is to provide the method of delivering subliminal messages to a user for providing positive benefits, wherein the subliminal messages comprises sound tracks, vocal messages; suggestions; inspirational speeches; graphics; text messages, or combination thereof.

Yet another object of the invention is to provide the method of delivering subliminal messages to a user for providing positive benefits, wherein the positive benefits comprises relieving the user from various diseases, improving the user's cognition health, relaxing the user, relieving anxiety, relieving stress, relieving pain, relieving grief, helping to quit alcohol or smoking, improving sexual health, increasing memory, improving weight loss results, controlling diabetes, controlling weight gain, increasing intelligence, bringing about behavioral changes, or similar type of positive benefits. Apart from providing relief from various ailments, the invention is helpful in learning new skills.

Yet another object of the invention is to provide the method of delivering subliminal messages to a user for providing positive benefits, wherein the subliminal messages have a frequency from 0.01 Hz-20 Hz.

Yet another object of the invention is to provide the method of delivering subliminal messages to a user for providing positive benefits, wherein the volume of the subliminal messages can be increased or decreased by the user.

Yet another object of the invention is to provide the method of delivering subliminal messages to a user for providing positive benefits, wherein the object device comprises smartphone, mobile phone, wearable device, video game consoles, wraparound device, hand held device, mobile station, vehicle computer system, PDA device, personal computer, computer, monitor, smart TVs, displays, laptop, notebook, internet pad, tablet, or the similar devices.

In this respect, before explaining at least one embodiment of the invention in detail, it is to be understood that the invention is not limited in its application to the details of construction and to the arrangements of the components set forth in the following description or illustrated in the drawing 3. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### (4) BRIEF DESCRIPTION OF THE DRAWING

The invention will be better understood and objects other than those set forth above will become apparent when consideration is given to the following detailed description thereof. Such description makes reference to the annexed drawings wherein:

FIG. 1 Illustrates an example by a flow chart of a method of delivering subliminal messages to a user for providing positive benefits.

### (5) DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural and logical changes may be made without departing from the spirit and scope of the present invention. The following detailed description is, therefore, not to be taken in a limiting sense, and the scope of the present invention is defined by the appended claims and their equivalents.
As used herein, the term “comprising” and its derivatives including “comprises” and “comprise” include each of the stated integers or elements but does not exclude the inclusion of one or more further integers or elements.
As used herein, the singular forms “a”, “an”, and “the” include plural referents unless the context clearly dictates otherwise. For example, reference to “a device” encompasses a single device as well as two or more devices, and the like.
As used herein, the terms “for example”, “like”, “such as”, or “including” are meant to introduce examples that further clarify more general subject matter. Unless otherwise specified, these examples are provided only as an aid for understanding the applications illustrated in the present disclosure, and are not meant to be limiting in any fashion.

The present invention relates to an injection method for subliminal messages in a device including mobile devices, computers, or similar devices including a memory element and a processor. The said subliminal message consists of messages in a specific frequency for various types that cannot be recognized, but perceived. The method can be applied to any kind of media the device is playing, no matter the played medium is screened in or out of the device with known means of the art. That is to say, for example the current invention can be applied in the method of sound if the device can run sound files or streams no matter on the device itself or an external speaker.

The invention relates to a method for playing subliminal messages in a device, via an installed application. The application pools subliminal messages of various types with a remote server. By this way, the subliminal messages can vary by time, type or frequency. The stored subliminal messages are sorted by the output type by known means of the art. So, there may be distinct messages for distinct types of data; there may be distinct types of subliminal messages for text-viewing, video, audio, etc.

Apart from pooled messages, this invention would include a format where user can type in the messages they would like to have and it would be read to them a subliminal sound level through a text to speech program.

The method is an injection method; the invention described herein applies subliminal messages during another content-viewing activity. That is to say, for example, during the time user plays an audio or video file or video stream, the present invention plays a subliminal message within. The user perceives no difference, but the present invention shows the demanded message.

The nature of the injection activity can be various according to the state of the content. According to one aspect of the invention, the present invention can co-stream another sound file with varying frequencies between 0.01 hz-20 hz

According to another aspect of the invention, the present invention, the present invention can stream a single-frame image within a video file or stream or video file. In this mode, the present invention removes a single frame from the original file, and replaces with the message to be displayed.

The messages in various frequencies or lengths cover contents consisting of subliminal, healing, binaural, brain wave, rife, etc. targeted messages. By these messages, the present invention aims weight loss, behavior change, money attraction or intelligence boosting. The aim and content of the message is designed within the generation of the message where the present invention is a method to play them in the desired manner. The aim of the subliminal message concept is to modify a person's behavior in the desired way.

The messages are not recognizable by the user in normal usage. But in an analysis (loud listening or slow-motion watching) conducted by an average user, the message is fully understandable. But under normal viewing or listening conditions, the user recognizes no difference from the original view of the content. Besides, the user is made subject to content by his own wish and selection within the application.

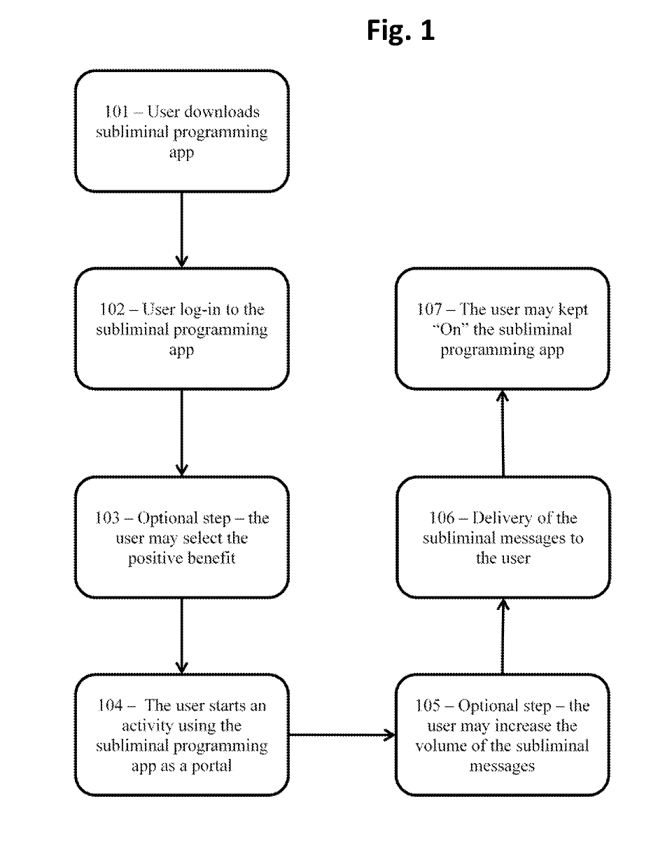

FIG. 1 illustrates an example by a flow chart of a method of delivering subliminal messages to a user for providing positive benefits. At first step 101, a user downloads subliminal programming app on an object device. The object device include smartphone, mobile phone, wearable device, virtual reality devices, VR glasses, video game consoles, wraparound device, hand held device, mobile station, vehicle computer system, PDA device, personal computer, computer, monitor, smart TVs, displays, laptop, notebook, internet pad, or the similar mobile or fixed devices. The subliminal programming app includes internet website based applications, server based applications, mobile applications or combination thereof. Further, the subliminal messages are audio subliminal messages.

At step 102, the user log-in to the subliminal programming app, as soon as the user log-in to the subliminal programming app, the application is in “On” mode. The user may have option of selecting one of the categories of the positive benefits (step 103). At step 104, the user can start any activity using the subliminal programming app as a portal. The activity may include web-surfing, playing video games, playing virtual reality games, visiting VR sites, playing the multimedia contents from the websites or thru the various mobile app. The websites or mobile app Pandora, Spotify, You tube, Apple Music, Facebook, Netflix, Amazon, Hulu or any similar websites or mobile app which provides audio and/or video contents. The user may have choice of adjusting the volume of the subliminal messages (step 105). The subliminal programming app starts delivering the subliminal messages (step 106). The user may have option of keeping “On” the subliminal messaging app. The subliminal messages can be audio subliminal messages. The subliminal programming app connects through the application programming interface (API) of the audio/video/game files of the websites or the mobile app. The subliminal programming app adds a second track which plays the audio subliminal messages. the subliminal messages comprises sound tracks, vocal messages; suggestions; inspirational speeches; graphics; text messages; musical tracks, or combination thereof. The frequencies of the subliminal messages are such that which cannot be heard human ear. The frequencies of the audio subliminal messages can be below 20 Hz. Sounds included are binural beats and brain wave frequencies 0.01 Hz-20 hz. Also included are higher frequencies, rife frequencies, healing frequencies, binural sounds, and delta, theta, alpha, and beta frequencies. The subliminal messages and suggestions are covering topics like weight loss or increasing intelligence, a skill a user wants to learn, or behavior they want to change such as quitting smoking, letting go of anxiety or stress, or attracting money. There are many studies about the effectiveness of subliminal programming and hypnosis such as visualize yourself playing tennis, running or exercising, drinking more water, or relieving pain and grief. As described above a method of delivering subliminal messages to a user for providing positive benefits. The positive benefits include relieving the user from various diseases, improving the user's cognition health, relaxing the user, relieving anxiety, relieving the stress, relieving pain, relieving grief, helping to quit alcohol, helping to quit smoking, improving sexual health, increasing memory, improving weight loss results, curing the diabetes, increasing intelligence, bringing behavioral changes, or similar type of positive benefits.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is claimed is:

1. A method of delivering audio subliminal messages using internet website based applications, server based applications, mobile applications or combination thereof, comprising of sound tracks, vocal messages; suggestions; inspirational speeches; text messages, or combination thereof, to a user for providing positive benefits that includes:
(i) Downloading a subliminal programming app to an object device;
(ii) Log-in to the subliminal programming app on the object device;
(iii) Optionally the user may also select one of the categories from the list of positive benefits;
(iv) the subliminal program plays full time when the person is doing internet activities. They do their normal web surfing subliminal the subliminal sounds are playing on a second track.
(v) starting web-surfing, start playing video games, for selecting choice of a website to play audio and/or video contents, for selecting choice of an mobile app to play audio and/or video contents, or combination thereof; and
(vi) Delivering the subliminal messages by the subliminal programming app on the background or on the secondary track to the user while he/she is performing the activity.

2) The method of delivering subliminal messages to a user for providing positive benefits according to claim 1, wherein the positive benefits comprises relieving the user from various diseases, improving the user's cognition health, relaxing the user, relieving anxiety, relieving the stress, relieving pain, relieving grief, helping to quit alcohol, helping to quit smoking, improving sexual health, increasing memory, improving weight loss results, controlling the diabetes, controlling the weight gain, increasing intelligence, making leaning in any field happening faster, learning a new foreign language, bringing behavioral changes, or similar type of positive benefits.

3. The method of delivering subliminal messages to a user for providing positive benefits according to claim 1, wherein the subliminal messages have a frequency of 0.01-20 Hz.

4. The method of delivering subliminal messages to a user for providing positive benefits according to claim 1, wherein the object device comprises smartphone, mobile phone, wearable device, virtual reality devices, VR glasses, video game consoles, wraparound device, hand held device, mobile station, vehicle computer system, PDA device, personal computer, computer, monitor, smart TVs, displays, laptop, notebook, internet pad, or the similar mobile or fixed devices.
